(19) United States
(12) Patent Application Publication
(10) Pub. No.: US 2022/0042969 A1
(43) Pub. Date: Feb. 10, 2022

(54) MASS CONTROL SYSTEM FOR CHROMATOGRAPHY

(71) Applicant: C TECHNOLOGIES, INC., Bridgewater, NJ (US)

(72) Inventors: Craig Harrison, Bridgewater, NJ (US); Ramsey Shanbaky, Bridgewater, NJ (US)

(73) Assignee: C TECHNOLOGIES, INC., Bridgewater, NJ (US)

(21) Appl. No.: 17/281,517

(22) PCT Filed: Oct. 8, 2019
(86) PCT No.: PCT/US2019/055179

§ 371 Date: Mar. 30, 2021

Related U.S. Application Data
(60) Provisional application No. 62/766,253, filed on Oct. 9, 2018.

(51) Int. Cl. G01N 33/487; B01D 15/38; G01N 30/02

(52) U.S. Cl. CPC G01N 33/48735; G01N 2030/027; G01N 30/02; B01D 15/38

(57) ABSTRACT

The present invention relates to methods for controlling chromatographic processes in real-time via mass measurement utilizing a variable pathlength spectrophotometer.

### RELATED APPLICATIONS

This application claims priority to U.S. Ser. No. 62/766,253 filed on Oct. 9, 2018, which is hereby incorporated into this application in its entirety.

### FIELD OF THE INVENTION

The present invention relates to methods for controlling chromatographic processes in real-time via mass measurement utilizing a variable pathlength spectrophotometer.

### BACKGROUND OF THE INVENTION

The chromatography process to purify a biomolecule is a cumbersome and time-consuming process. It requires equipment capable of monitoring UV absorbance, conductivity, pH, flow rate and other parameters. Affinity chromatography is commonly the first chromatography step in the purification process and is where the protein of interest is mostly separated from the complex mixture of harvested cell culture fluid or fermentation harvest. The amount of material loaded on a column, flow rate of the material over the column and column size or bed height defines the residence time of the material in the column. Residence time has a direct relationship to dynamic binding capacity (GE paper). The dynamic binding capacity of a chromatography media is the amount of target protein the media will bind under actual flow conditions before significant breakthrough of unbound protein occurs. For any given residence time there is breakthrough curve associated with the dynamic binding capacity. The dynamic binding capacity reflects the impact of mass transfer limitations that may occur as flow rate is increased and is more useful in predicting real process performance than a determination of saturated or static capacity. The breakthrough curve in an affinity chromatography process describes the percentage of material leaving the column and not being bound. In order to design an efficient and useful process the appropriate residence time, loading and number of cycles for a given batch depending on the amount of mass that must be processed should be determined. In general, dynamic capacity will decrease as residence time decreases, however the rate at which the dynamic capacity decreases can vary greatly from medium to medium. An ideal medium would have efficient mass transfer properties across the range of flow rate, but in practice there is an upper limit to the flow rate that is determined by the mechanical strength of the medium. Optimization of the process criteria for maximum dynamic binding capacity leads to less need for excess process scale-up as well as decreased process time, costs and protein loss. This is the case for even a single column chromatography step and is complicated when continuous chromatography utilizing several columns is used in the purification process. In cases where the feed concentrations and/or flow rates vary with time or if the column materials are different the dynamic binding capacity will be different or will change with time. Moreover, usage the material in the column will change over time and the process conditions used when the column is new will be different than when the column is older. Therefore, there is a need to provide real time information concerning the dynamic binding capacity at a given break through level and as well as protein titer and mass information.

Rather than using single pathlength UV absorbance sensors that have a limited linear range, a variable pathlength UV spectrophotometer is utilized. Since the variable pathlength spectrophotometer can provide a slope value in absorbance/mm that can be easily and accurately converted to concentration of the protein using the extinction coefficient (mL/cm*mg), an accurate mass can be calculated.

### SUMMARY OF THE INVENTION

In the past a single pathlength UV absorbance sensor that has limited linear range was used to determine chromatography parameters. In the present invention, a variable pathlength UV spectrophotometer is utilized since the variable pathlength spectrophotometer can provide a slope value in absorbance/mm that can be easily and accurately converted to concentration of the protein using the extinction coefficient (mL/cm*mg), an accurate mass can be calculated.

The present invention relates to methods for determining the breakthrough percentage of a chromatography column by determining the initial slope using slope spectroscopy for a given protein (m0) by flowing a harvested cell culture fluid through the chromatography column for enough time to establish a signal that is not changing and determining the a first slope (m1) by positioning a sensor at the inlet to the column and measuring the slope by slope spectroscopy and determining a second slope (m2) by positioning a sensor at the outlet to the column and measuring the slope by slope spectroscopy and the calculating the breakthrough percentage by calculating % BT=(m2−m0)/(m1−m0)*100.

The present invention also relates to methods for determining the protein titer of a chromatography column by determining the initial slope (m0) by flowing a harvested cell culture fluid through the chromatography column for enough time to establish a signal that is not changing wherein the initial slope is determine by slope spectroscopy and then determining a first slope (m1) by positioning a sensor at the inlet to the column and measuring the slope by slope spectroscopy and then calculating the titer of the chromatography column by calculating Titer=(m1−m0)/EC wherein EC is the extinction coefficient of the protein in units of mL/mg*cm.

The present invention relates to methods for determining the real-time mass of a protein loaded on a chromatography column comprising by determining the protein titer of the chromatography column as described above and calculating the real-time mass of a protein loaded on a chromatography by calculating mass column 1 (mg)=Titer*flow rate*time.

The present invention relates to methods for determining the real-time mass of a protein loaded onto a second chromatography column in a chromatography process having two chromatography columns comprising determining the percentage breakthrough of the first chromatography column as described above and calculating the real-time mass of a protein loaded on the second chromatography by calculating mass column 2 (mg)=% BT*titer*flow rate*time

Similar types of control schemes can be utilized for subsequent polishing steps such as anion exchange, cation exchange or mixed mode chromatography.

### DETAILED DESCRIPTION OF THE INVENTION

Electromagnetic radiation (light) of a known wavelength, λ, (ie. ultraviolet, infrared, visible, etc.) and intensity (I) is incident on one side of the cuvette. A detector, which measures the intensity of the exiting light, I is placed on the opposite side of the cuvette. The length that the light propagates through the sample is the distance d. Most standard UV/visible spectrophotometers utilize standard cuvettes which have 1 cm path lengths and normally hold 50 to 2000 μm of sample. For a sample consisting of a single homogeneous substance with a concentration c, the light transmitted through the sample will follow a relationship know as Beer's Law: A=εcl where A is the absorbance (also known as the optical density (OD) of the sample at wavelength λ where OD=the −log of the ratio of transmitted light to the incident light), ε is the absorptivity or extinction coefficient (normally at constant at a given wavelength), c is the concentration of the sample and I is the path length of light through the sample.

Often the compound of interest in solution is highly concentrated. For example, certain biological samples, such as proteins, DNA or RNA are often isolated in concentrations that fall outside the linear range of the spectrophotometer when absorbance is measured. Therefore, dilution of the sample is often required to measure an absorbance value that falls within the linear range of the instrument. Frequently multiple dilutions of the sample are required which leads to both dilution errors and the removal of the sample diluted for any downstream application. It is, therefore, desirable to take existing samples with no knowledge of the possible concentration and measure the absorption of these samples without dilution. In a continuous process such as protein purification the one or more flow sensors of the present invention could be utilized at each step of the process or at particular sites in the process. In step 1 of the process the harvest material is a combination of the target protein, host cell proteins, media, DNA and other impurities. A slope signal would give the absorbance contributions of all these components. With characterization it may be possible to use a spectral signal to quantify components. The spectra could be used as a pre-column indicator to compare to a post column slope signal to determine column loading in either a batch or continuous process. Alternatively, using a slope signal before and after the column the product titer can be determined. Once the product titer is compared to the concentration signal a real-time mass during loading can be determined. This allows for the material prior to the column contains the full complement of loading materials. Once the column is loaded the target protein is adsorbed or bound to the column and the material flowing through the column are the impurities from the harvested material. Conversely in an exclusion column would capture the impurities and permit the target material to pass through the column. The second step of the process, after the affinity column, may be the best location to monitor the process. This step is where most of the purification of the substance occurs. A slope signal can be used to see when a column is fully loaded. This may be accomplished by a comparison of the background signal (due to the harvest material alone) as it is flowing past the sensor to a signal at a later time of the harvest material and load material together. This occurs when the resin is loaded to capacity. Alternatively, by having the product titer and real-time concentration, loading on a column can be controlled by mass of total protein loaded. Parameters like pH, flow rate, conductivity, size and configuration of resin, type of resin or temperature may affect the loading capacity. With this slope signal alone, load capacity may be determined quickly and varied experimentally to hone in on ideal process parameters. During a continuous process, there would likely be many affinity columns that would individually be loaded to capacity and then eluted. Long-term comparison of elution peak from column to column could indicate if resin capacity has dropped over time indicating a need to replace a column or other change in the process. The addition of spectral measurements during elution may allow for quantification of individual components present in the solution. Steps 3 and 4 are polishing steps and a slope sensor at each polishing step provides a continuous quantification of the concentration and an overall yield value for the process. Due to the large dynamic range of the flow sensors multiple species can be quantified in ion exchange chromatography separation which otherwise would take offline analysis. In step 5 a sensor after the UF/DF stage gives a concentration value that is the final concentration of the drug substance which has been processed/purified. The concentration can be monitored throughout the process easily without extensive characterization which contrasts other methods like refractive index monitoring. Slope value is in most cases buffer independent. The permeate can also be monitored during normal processing or conjugation. In the final step flow sensor at the filling station will give a final vial concentration. It can be used to capture all remaining material and be used to determine final process yield. While In many embodiments of the methods of the present invention a single wavelength may be monitored it may be advantageous in certain circumstances to monitor two or more wavelengths. For example over time a contaminant in the product line may build up such that the contaminant deposit such that eventually the light to the detector become partially or fully occluded. Monitoring an off-peak wavelength during a continuous process could detect this issue prior to it becoming a problem.

A variable path length spectrophotometer which dynamically adapts parameters in response to real time measurements via software control to expand the dynamic range of a conventionally spectrophotometer such that samples of almost any concentration can be measured without dilution or concentration of the original sample. Furthermore, methods of the present invention do not require that the path length be known to determine the concentration of samples.

The methods of the present invention provide a novel technique of determining loading mass by establishing an initial slope in Abs/mm (m0) during the loading curve and subtracting it from the slope before and after the chromatography column. The flow rate (mL/min) and extinction coefficient are then applied and integrated in real-time to determine the mass loaded on the column and/or subsequent columns. In this invention, a combination of 1 or 2 sensors are used. In the scheme with 2 sensors, one is placed at the inlet of the column that generates the first slope value(m1, Abs/mm) and one is placed at the outlet of the column for the 2^{nd} slope value(m2, Abs/mm). A combination of an offline slope measurement of the inlet can be used in lieu of m1. The initial slope (m0) is determined by flowing the harvested cell culture fluid (HCCF) through the column for enough time to establish a signal that remains relatively unchanged for a period of time. This volume is typically determined after the flow of at least 1-2 column volumes through the column. It may take as much as much as 4 column volumes (CVs) through the column before the signal stabilizes. After the m0 slope (Abs/mm) is established, this value can be input into the control system to start plotting % breakthrough (% BT) vs. time.

% BT=(m2−m0)/(m1−m0)*100

Protein titer can also be determined as:

Titer=(m1−m0)/EC

Titer in units of mg/m1, m1 and m0 in Abs/mm and EC in mL/mg*cm

The real-time mass loaded on the column is

Mass column 1 (mg)=Titer*flow rate*time

The real-time mass loaded on a subsequent column is

Mass column 2 (mg)=% BT*titer*flow rate*time

This control scheme can be used in single column or multicolumn affinity chromatography. In single column chromatography, the mass control allows maximum loading on a column. The use of the methodology will provide an increase in flexibility and control of a batch process. Resin degradation no longer need be accounted for because the control system adapts to any binding capacity.

In a multi-column process, mass control provides the loading of the first and 2^{nd} column in real-time. This control system can then adapt to perfusion bioreactors where the titer may be dynamic. Timing can be determined quickly and accurately by having a mass control system. In connected batch multi-column processes it provides a similar advantage as a single column.

A flow-through device may serve as a vessel for the measurements made in the methods of the present invention. The flow-through device comprises a flow cell body that permits the flow of a sample solution into and out of the flow cell device. The flow cell body has at least one window that is transparent to electromagnetic radiation in the range of electromagnetic source typically 200-1100 nm. The window can be made from various materials but for ultraviolet applications quartz, cyclo olefin polymer (COP), cyclo olefin copolymer (COC), polystyrene (PS) or polymethyl methacrylate PMMA may be required. The window may be of different sizes and shapes so long as the electromagnetic radiation can pass through the window and strike the detector. In a flow-cell system the detector and probe tip may be in a substantially horizontal orientation and the sample flows between the detector and the probe. In an alternate embodiment a mirror may be used to reflect the electromagnetic radiation to and through the window. The placement of the mirror and window are not restricted as long as the mirror can reflect the electromagnetic radiation through the window such that the radiation is detected by the detector. In certain embodiments the mirror and the window may be opposite one another or at right angles to each other. Regardless of the absolute spatial orientation of the probe and detector, the probe tip and surface of the detector should be substantially perpendicular relative to one another. The flow cell body also comprises a port through which the probe tip may pass. This port is sealed with a dynamic seal such that the probe tip can pass through the port without sample solution leaking from the flow-through device. Such seals include FlexiSeal Rod and Piston Seals available from Parker Hannifin Corporation EPS Division, West Salt Lake City, Utah. In the diagram there is a single pathway for the sample solution to flow coming in the inlet port and exiting the outlet port. Alternative embodiments may include multiple pathways and multiple inlet and outlet ports. In the flow cell device, the probe tip moves substantially perpendicular to the flow of the sample solution and is substantially perpendicular to the detector. The flow cells may have a variety of inside diameters. The various flow cell diameters are a function of the volume and flow rate needed during a given process.

The flow cells may be incorporated into the flow stream by various fittings. The 3 mm ID flow cell uses a barb fitting or luer fitting. The 10 mm ID flow cell uses a tri-clamp fitting. In a preferred embodiment of the flow cell, the cells are made of stainless steel 316, with a quartz window and a fiber optic encased in stainless. In this preferred embodiment there are 2 teflon seals on either side of the fibrette that pistons up and down in the flow cell in order to take reading. Alternatively, a gasket fixed to the fibrette and fixed in the flow cell can provide the proper sealing while ensuring accurate path length changes. In preferred embodiments of the flow cell the outer diameter of the fibrette is increased compared to static systems. In preferred embodiments the outer diameter of the fibrette may be less than 1 mm or greater than 25 mm. The size of the fibrette will depend on the application which will influence the size of the flow cell and the rate of the fluid flowing through the flow cell. In preferred embodiments the fibrette is of sufficient diameter so that it will not vibrate, bend or break. The increased outer diameter of the fibrette reduces equipment vibration that impacts the accuracy of the measurement. In a preferred embodiment of the flow cell there is a stainless plug located between the Teflon seals. The plug fills a void in the flow cell that may present a cleaning challenge. With the void filled, the flow cell is more easily cleaned. Other seals in the flow cell may be made with platinum cured silicone. Standard EPDM seals may release some material over time that may contaminate the flow cell and the use of platinum cured silicone avoids this potential issue. The flow cells of the present invention are capable of being sterilized or cleaned such that they may be used in processes where a sterile or aseptic environment is required.

Detectors comprise any mechanism capable of converting energy from detected light into signals that may be processed by the device. Suitable detectors include photomultiplier tubes, photodiodes, avalanche photodiodes, charge-coupled devices (CCD), and intensified CCDs, among others. Depending on the detector, light source, and assay mode such detectors may be used in a variety of detection modes including but not limited to discrete, analog, point or imaging modes. Detectors can used to measure absorbance, photoluminescence and scattering. The devices of the present invention may use one or more detectors although in a preferred embodiment a single detector is used. In a preferred embodiment a photomultiplier tube is used as the detector. The detectors of the instrument of the present invention can either be integrated to the instrument of can be located remotely by operably linking the detector to a light delivery device that can carry the electromagnetic radiation the travels through the sample to the detector. The light delivery device can be fused silica, glass, plastic or any transmissible material appropriate for the wavelength range of the electromagnetic source and detector. The light delivery device may be comprised of a single fiber or of multiple fibers and these fibers can be of different diameters depending on the utilization of the instrument. The fibers can be of almost any diameter but in most embodiments the fiber diameter is in the range of from about 0.005 mm to about 20.0 mm.

The control software will adapt the devices behavior based upon various criteria such as but not limited to wavelength, path length, data acquisition modes (for both wavelength/path length), kinetics, triggers/targets, discrete path length/wavelength bands to provide different dynamic ranges/resolutions for different areas of the spectrum, cross sectional plot to create abs/path length curves, regression algorithms and slope determination, concentration determination from slope values, extinction coefficient determination, base line correction, and scatter correction. The software is configured to provide scanning or discrete wavelength read options, signal averaging times, wavelength interval, scanning or discrete path length read options, data processing option such as base line correction, scatter correction, real-time wavelength cross-section, threshold options (such as wavelength, path length, absorbance, slope, intercept, coefficient of determination, etc.) an kinetic/continuous measurement options.

1. A method for determining the breakthrough percentage of a chromatography column having an inlet and an outlet comprising:
determining an initial slope (m0) by flowing a harvested cell culture fluid through the chromatography column for enough time to establish a signal that is not changing wherein the initial slope is determine by slope spectroscopy;
determining a first slope (m1) by slope spectroscopy with a first sensor positioned at the inlet to the chromatography column;
determining a second slope (m2) by slope spectroscopy with a second sensor positioned at the outlet to the chromatography column; and
determining the breakthrough percentage by calculating % BT=(m2−m0)/(m1−m0)*100.

2-4. (canceled)

5. The method of claim 1, wherein the method is used to determine the breakthrough percentage of the chromatography column in a continuous process.

6. The method of claim 1, further comprising calculating a real-time mass of a protein loaded onto the chromatography column (mg) as Titer*flow rate*time.

7. The method of claim 1, further comprising:
determining a second column initial slope (m₂0) by flowing a harvested cell culture fluid through a second chromatography column for enough time to establish a second column signal that is not changing wherein the second column initial slope is determine by slope spectroscopy;
determining a second column first slope (m₂1) by slope spectroscopy with a second column first sensor positioned at a second column inlet of the second chromatography column;
determining a second column second slope (m₂2) by slope spectroscopy with a second column second sensor positioned at a second column outlet of the second chromatography column; and
determining a second breakthrough percentage by calculating % BT₂=(m₂2−m₂0)/(m₂1−m₂0)*100, calculating a real-time mass of a protein loaded on the second chromatography column (mg) as % BT₂*titer₂*flow rate₂*time, or both.

8. The method of claim 7, further comprising comparing a first elution peak of the chromatography column to a second elution peak of the second chromatography column.

9. The method of claim 8, further comprising determining that at least one of the chromatography column or the second chromatography column should be replaced based on a decrease of a resin capacity over time.

10. The method of claim 1, further comprising determining a load capacity of the chromatography column based on the breakthrough percentage, determining if the chromatography column is fully loaded based on the breakthrough percentage, or both.

11. The method of claim 1, further comprising determining an optimization of at least one of a pH level, flow rate, conductivity, size of resin, configuration of resin, type of resin, or temperature associated with the chromatography column based on the breakthrough percentage.

12. The method of claim 1, wherein determining the first slope with the first sensor, determining the second slope with the second sensor, or both, comprises determining a continuous quantification of a concentration of at least one species of interest in the harvested cell culture over time.

13. The method of claim 1, further comprising determining a titer of the chromatography column by calculating Titer=(m1−m0)/EC wherein EC is an extinction coefficient of a protein in units of mL/mg*cm.

14. A method for determining a protein titer of a chromatography column having an inlet and an outlet comprising:
determining an initial slope (m0) by flowing a harvested cell culture fluid through the chromatography column for enough time to establish a signal that is not changing wherein the initial slope is determine by slope spectroscopy;
determining a first slope (m1) by positioning a sensor at the inlet to the column and measuring the slope by slope spectroscopy; and
determining a titer of the chromatography column by calculating Titer=(m1−m0)/EC wherein EC is an extinction coefficient of a protein in units of mL/mg*cm.

15. The method of claim 14, further comprising calculating a real-time mass of a protein loaded on a chromatography by calculating mass column 1 (mg)=Titer*flow rate*time.

16. The method of claim 14, wherein the method is used to determine a breakthrough percentage of the chromatography column in a continuous process.

17. The method of claim 14, further comprising calculating a breakthrough percentage by calculating % BT=(m2−m0)/(m1−m0)*100.

18. A method for determining a real-time mass of a protein loaded onto a second chromatography column in a chromatography process comprising a first and a second chromatography column comprising:
determining the titer of the first chromatography column according to claim 14; and
calculating the real-time mass of a protein loaded on the second chromatography column (mg) as % BT*titer*flow rate*time.

19. The method of claim 18, further comprising comparing a first elution peak of the chromatography column to a second elution peak of the second chromatography column.

20. The method of claim 18, further comprising determining that at least one of the chromatography column or the second chromatography column should be replaced based on a decrease of a resin capacity over time.

21. The method of claim 14, further comprising determining a load capacity of the chromatography column or a second chromatography column based on the respective titer.

22. The method of claim 14, further comprising determining an optimization of at least one of a pH level, flow rate, conductivity, size of resin, configuration of resin, type of resin, or temperature associated with the chromatography column based on the titer.

23. The method of claim 14, wherein determining the first slope with the sensor, determining a second slope with a second sensor, or both, comprises determining a continuous quantification of a concentration of at least one species of interest in the harvested cell culture fluid over time.
